(19) United States
(12) Patent Application Publication
(10) Pub. No.: US 2021/0012859 A1
(43) Pub. Date: Jan. 14, 2021

(54) Method For Determining Genotypes in Regions of High Homology

(71) Applicant: MYRIAD WOMEN'S HEALTH, INC., SOUTH SAN FRANCISCO, CA (US)

(72) Inventors: Dale E. MUZZEY, San Francisco, CA (US); Eric A. EVANS, San Bruno, CA (US); Jared R. MAGUIRE, San Francisco, CA (US); Alexander D. ROBERTSON, South San Francisco, CA (US)

(73) Assignee: MYRIAD WOMEN'S HEALTH, INC., SOUTH SAN FRANCISCO, CA (US)

(21) Appl. No.: 16/944,048

(22) Filed: Jul. 30, 2020

Related U.S. Application Data
(63) Continuation of application No. 14/979,519, filed on Dec. 28, 2015.
(60) Provisional application No. 62/097,139, filed on Dec. 29, 2014.
(60) Provisional application No. 62/234,012, filed on Sep. 28, 2015.

(51) Int. Cl. G16B 20/00; C12Q 1/6883; C12Q 1/6869

(52) U.S. Cl. CPC G16B 20/00; C12Q 1/6883; G16B 30/00; C12Q 2600/156; C12Q 1/6869

(57) ABSTRACT

Described herein are methods directed to determining the carrier status or genotype of a subject. Described herein is a method that combines experimental and computational approaches to resolve the structure of genomic loci (i.e., the genotype) whose sequences are highly homologous to other sequences in the genome. In particular, the determination of carrier status and/or copy number of a gene in a subject, wherein the gene has a corresponding highly homologous homolog, e.g., gene or pseudogene, utilizes Next Generation Sequencing. Also described herein is a computer-assisted method for such determinations.

### CROSS-REFERENCE TO RELATED APPLICATIONS

The present application is a continuation of U.S. patent application Ser. No. 14/979,519, filed Dec. 28, 2015, which claims priority to U.S. Provisional Application No. 62/097,139, filed Dec. 29, 2014, and to U.S. Provisional Application No. 62/234,012, filed Sep. 28, 2015, each of which are incorporated herein by reference.

### TECHNICAL FIELD

The following disclosure relates generally to determining genotypes and, more specifically, to determining genotypes associated with a gene having a corresponding highly homologous homolog.

### BACKGROUND

Many diseases result from genes rendered inactive by mutation. Identification of such mutations is, therefore, a fundamental goal of clinical genetic medicine. For many genes, these mutations are relatively easy to find from Next Generation Sequencing (NGS) data. However, for a subset of genes that are the subject of several important and prevalent disorders, it is challenging to identify and count the number of inactivated genes, since these genes are effectively occluded by other homologous parts of the genome.

Resolving the structure and content of genomic regions that are highly homologous to other (typically dysfunctional) regions is exceptionally difficult, even with advanced NGS tools. Unfortunately, these technical obstacles are especially problematic, as many of these difficult regions have disease implications. Indeed, their very homology to dysfunctional regions leads to frequent rearrangements between genes and homologs, which can affect the number of functional copies of the gene.

Thus, there remains a need for detecting and determining the genotype and/or carrier status of a subject with respect to a gene, wherein the gene has a homologous homolog.

### BRIEF SUMMARY OF THE INVENTION

Current technologies that allow determination of genotypes for highly homologous genes and the corresponding homologs are time- and labor-intensive, as well as expensive, making them unsuitable for widespread clinical use.

The presently disclosed methods may be practiced in an affordable and high-throughput manner. Thus, there are significant time, labor and expense savings. In addition, the present method overcomes the problem of resolving structure/copy-number/genotype in regions where the unique alignment of NGS reads to genes or their homologs is compromised. Importantly, these compromising “highly homologous” regions are based on two features: (1) the length of the NGS reads in the given experiment and (2) the amount of mismatches allowed by the alignment software, e.g., BWA.

In an aspect there is provided herein a method for determining the genomic structure (i.e., genotype) of an individual with respect to a gene of interest, wherein the gene of interest has a highly homologous homolog.

In an embodiment the sequence information for the gene of interest and its homolog use primers that are directed to an exon. In certain embodiments, the sequence information is from an intron of a gene of interest and/or homolog. In certain embodiments, the sequence information is from intergenic regions.

In a further embodiment, the sequence information is generated by Next Generation Sequencing (NGS). In some embodiments the NGS is high-depth whole-genome shotgun sequencing without the use of probes for enrichment). In other embodiments, the NGS is targeted sequencing such as, for example, hybrid-capture technology, multiplex amplicon enrichment, or any other means of enriching specific regions of the genome for the sequencing reaction. In some embodiments, the sequencing is done in a multiplex assay.

In one embodiment, the gene is SMN1 and the pseudogene is SMN2. In an embodiment, the presence of an altered copy number of SMN1 indicates that the subject may be a carrier for the disease spinal muscular atrophy (SMA).

In another embodiment, the gene is CYP21A2 and the pseudogene is CYP21A1P. In an embodiment, the presence of an altered copy number of CYP21A2 indicates that the subject may be a carrier for the disease congenital adrenal hyperplasia (CAH).

In an embodiment, the gene is HBA1 and the homolog is HBA2 (or vice versa). In an embodiment, the presence of an altered copy number of either HBA1 or HBA2 indicates that the subject may be a carrier for the disease alpha-thalassemia.

In a further embodiment, the gene is GBA and the pseudogene is GBAP. In an embodiment, the presence of an altered copy number of GBA indicates that the subject may be a carrier for the disease Gaucher's Disease.

In an embodiment, the gene is PMS2 and the pseudogene is either PMS2CL or one of several other pseudogenes. As of December 2015 there were 15 pseudogenes. The pseudogenes may be selected from, but not limited to, the 13 pseudogenes known as PMS2CL with the other 12 of 13 pseudogenes numbered PMS2P1 through PMS2P12. In an embodiment, the presence of an altered copy number and/or inversions that alter orientation of the gene and pseudogene (e.g., those that fuse portions of pseudogene with the gene and thus compromise gene function) may indicate that the subject has increased risk for the disease Lynch Syndrome.

In an embodiment, the gene is CHEK2, which has several pseudogenes. As of Dec 2014, here were seven pseudogenes. The pseudogenes may be selected from, but not limited to, CHEK2 pseudogenes enumerated in a curated database. In an embodiment, the presence of mutations that arise from recombination with its pseudogenes—e.g., a pseudogene-derived frameshift mutation—may indicate that the subject has increased risk for the disease breast cancer, among other diseases. It is well known in the art that only one of the seven pseudogenes has been named and that risk is primarily associated with one mutation, 1100delC. However, other mutations also contribute to risk of disease. Patients are at risk for Li Fraumeni syndrome and other heritable cancers.

In an aspect, there is provided a computer system configured to execute instructions for carrying out the methods described herein.

Other objects, features and advantages of the present invention will become apparent from the following detailed description. It should be understood, however, that the detailed description and specific examples, while indicating preferred embodiments of the invention, are given by way of illustration only, since various changes and modifications within the scope and spirit of the invention will become apparent to one skilled in the art from this detailed description.

### BRIEF DESCRIPTION OF THE DRAWINGS

FIG. 1 illustrates various genomic structures of genes and their homologs (e.g., dysfunctional homologs in the case of pseudogenes). In a “normal” sample, there are two copies each of the gene and its homolog. For many genes with homologs—indeed for the genes that underlie Gaucher's Disease, Spinal Muscular Atrophy (“SMA”), Congenital Adrenal Hyperplasia (“CAH”), and alpha-thalassemia, as well as several genes linked to various cancers—the gene and homolog are in relatively close proximity to each other on the chromosome. Some examples of chromosomes that have undergone “deletion or duplication” of the gene and/or homolog are shown. Recombination between the gene and homolog can yield “fusion” genes that are part “gene” and part “homolog”. Finally, “interchange” of sequences between gene and homolog is relatively frequent.

FIG. 2 is a flow chart of a method as described herein.

FIG. 3 illustrates an exemplary system and environment in which various embodiments of the invention may operate.

FIG. 4 illustrates an exemplary computing system.

FIG. 5 is a copy number (“CN”) graph of SMN1 and SMN2. For 10,000 samples, we used sequencing data and the CN analysis described herein to calculate the sample's CN of SMN1 and SMN2 and then used these values as the x- and y-coordinates, respectively, in the scatterplot. The CN(SMN1), i.e., the copy number of SMN1, of each sample was validated by an orthogonal qPCR-based assay: samples determined by this latter assay to have 1, 2, or 3 copies are indicated by circles, triangles, and squares, respectively. Note that there is very clear separation in the sequencing data between the points with CN(SMN1)=1 and CN(SMN1)=2. Indeed, using a cutoff of CN(SMN1)=1.4 to classify samples as having either 1 or 2 copies of SMN1, our sequencing-based CN analysis would yield no false positives or false negatives. Other noteworthy features or the plot include: (1) the highest density of points is near (2,2), which is the normal arrangement of the locus; (2) many samples, however, are far from (2,2), consistent with frequent conversion/deletion/duplication between SMN1 and SMN2.

FIG. 6 shows two copy number graphs for GBA and GBAP. For two single patient samples, CN values for GBA and its homolog/pseudogene GBAP are plotted at nine different sites, arranged from 5′ to 3′ (left to right). The top sample (A) is normal since it has two copies of both GBA and GBAP. However, the bottom sample (B) has undergone an “interchange” event, where the 3′ end of one of the GBAP copies has acquired GBA-derived sequence.

FIG. 7 is a copy number graph for HBA1 and HBA2. The plot shows CN values for 48 patient samples in the area surrounding and including HBA2 and HBA1. The thick line shows a single sample in which a large segment of a single chromosome has been deleted, hence its drop in signal for much of the right side of the figure. As expected, most of the samples have CN=2. Three samples have short deletions that occur between the Z1 and Z2 regions.

FIG. 8 is a graph that shows the copy number for each probe used in the CYP21A2 gene and its homolog CYP21A1P. The plots show CN values for 48 patient samples in the gene CYP21A2 (A; left)—which affects CAH—and its pseudogene CYP21A1P (B; right). Each position on the x-axis is a different site in the gene, arranged from 5′ to 3′. The three thick traces are samples that are known to have undergone fusion events that ablate one of the copies of the gene, hence their CN values of ˜1 and ˜0 in the gene plot at left. CYP21A2 and CYP21A1P have undergone considerable interchange/fusion/deletion/duplication throughout evolution, which is why their traces in the plots above are more jagged than the CN traces in prior figures for Gaucher's Disease (FIG. 6) and alpha-thalassemia (FIG. 7). Note that one of the key goals of the CN analysis method described herein is that we want to determine the number of functional gene copies CYP21A2 in this case). As such, we first look at sites proximal to the 5′ end and use their average value to resolve CN(CYP21A2). Next, we consider the entirety of the trace (i.e., including the 3′ end) to determine what types of rearrangements have occurred.

FIG. 9 is a figure illustrating how the sample data gets processed from raw read counts into values that may be interpreted for copy-number shifts. Shown are six steps and five exemplary tables (designated a, b, c, d and e) that are further described herein, infra.

The file of this patent contains at least one drawing in color. Copies of this patent or patent publication with color drawing(s) will be provided by the Office upon request and payment of the necessary fee.

### DETAILED DESCRIPTION

The invention will now be described in detail by way of reference only using the following definitions and examples. All patents and publications, including all sequences disclosed within such patents and publications, referred to herein are expressly incorporated by reference.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this invention. Although any methods and materials similar or equivalent to those described herein can be used in the practice or testing of the present invention, the preferred methods and materials are described. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

Numeric ranges are inclusive of the numbers defining the range. The term “about” is used herein to mean plus or minus ten percent (10%) of a value. For example, “about 100” refers to any number between 90 and 110.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

### Definitions

As used herein, “purified” means that a molecule is present in a sample at a concentration of at least 95% by weight, or at least 98% by weight of the sample in which it is contained.

An “isolated” molecule is a nucleic acid molecule that is separated from at least one other molecule with which it is ordinarily associated, for example, in its natural environment. An isolated nucleic acid molecule includes a nucleic acid molecule contained in cells that ordinarily express the nucleic acid molecule, but the nucleic acid molecule is present extrachromasomally or at a chromosomal location that is different from its natural chromosomal location.

The term “% homology” is used interchangeably herein with the term “% identity” herein and refers to the level of nucleic acid or amino acid sequence identity between the nucleic acid sequence that encodes any one of the inventive polypeptides or the inventive polypeptide's amino acid sequence, when aligned using a sequence alignment program. In the case of a nucleic acid the term also applies to the intronic and/or intergenic regions.

For example, as used herein, 80% homology means the same thing as 80% sequence identity determined by a defined algorithm, and accordingly a homolog of a given sequence has greater than 80% sequence identity over a length of the given sequence. Exemplary levels of sequence identity include, but are not limited to, 80, 85, 90, 95, 98% or more sequence identity to a given sequence, e.g., the coding sequence for any one of the inventive polypeptides, as described herein.

Exemplary computer programs which can be used to determine identity between two sequences include, but are not limited to, the suite of BLAST programs, e.g., BLASTN, BLASTX, and TBLASTX, BLASTP and TBLASTN, and BLAT publicly available on the Internet. See also, Altschul, et al., 1990 and Altschul, et al., 1997.

Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. (See, e.g., Altschul, S. F., et al., Nucleic Acids Res. 25:3389-3402, 1997.)

A preferred alignment of selected sequences in order to determine “% identity” between two or more sequences, is performed using for example, the CLUSTAL-W program in MacVector version 13.0.7, operated with default parameters, including an open gap penalty of 10.0, an extended gap penalty of 0.1, and a BLOSUM 30 similarity matrix.

As used herein, “highly homologous” means that the homology between a gene and its corresponding homolog is greater than 90% over a region whose length corresponds to the NGS read length. Thus, a gene and its homolog are referred to as “highly homologous” if any region in the gene is highly homologous to the homolog. An NGS read length may range from 30 nt to 400 nt, from 50 nt to 250 nt, from 50 nt to 150 nt, or from 100 nt to 200 nt. Importantly, the entire gene's sequence need not be “highly homologous” to say a gene has a homolog; only a region in the gene needs to be highly homologous.

The term “homolog” as used herein refers to a DNA sequence that is identical or nearly identical to a gene of interest located elsewhere in the subject's genome. The homolog can be either another gene, a “pseudogene,” or a segment of sequence that is not part of a gene.

The term “mutation” as used herein refers to both spontaneous and inherited sequence variations, including, but not limited to, variations between individuals, or between an individual's sequence and a reference sequence. Exemplary mutations include, but are not limited to, SNPs, indel, copy number variants, inversions, translocations, chromosomal fusions, etc.

A “pseudogene” as used herein is a DNA sequence that closely resembles a gene in DNA sequence but harbors at least one change that renders it dysfunctional. The change may be a single residue mutation. The change may result in a splice variant. The change may result in early termination of translation. A pseudogene is a dysfunctional relative of a functional gene. Pseudogenes are characterized by a combination of homology to a known gene (i.e., a gene of interest) and nonfunctionality.

The number of pseudogenes for genes is not limited to those enumerated herein. Pseudogenes are increasingly recognized. Therefore, a person skilled in the art would be able to determine if a sequence is a pseudogene on the basis of sequence homology or by reference to a curated database such as, for example, GeneCards (genecards.org), pseudogenes.org, etc.

As used herein, a “gene of interest” is a gene for which determining the number of functional copies is desired. Generally, a gene of interest has two functional copies due to the two chromosomes each having a copy of the gene of interest. The terms “gene of interest” and “gene” may be used interchangeably herein,

### Process

Sequences from the region of interest are enriched, where possible, with hybrid-capture probes or PCR primers, which should be designed such that the captured and sequenced fragments contain at least one sequence that distinguishes a gene from its homolog(s). For example, hybrid-capture probes may be designed to anneal adjacent to the few bases that differ between the gene and the homolog(s)/pseudogene(s) (“diff bases”). Where such distinguishing sequence is scarce, multiple probes should be used to capture distinguishable fragments to diminish the effect of biases inherent to each particular probe's sequence. Amplicon sequencing can be used as an alternative to hybrid-capture as a means to achieve targeted sequencing, High-depth whole-genome sequencing can be used as an alternative to targeted sequencing. Any high-throughput quantitative data that reflects the dose of a particular genomic region may be used, be it from NGS, microarrays, or any other high-throughput quantitative molecular biology technique.

The abundance of NGS sequence reads bearing gene- or homolog-derived bases permit distinction between normal (CN=2) and mutant individuals (CN≠2). Additional useful information is attainable, however, even from sequence reads that cannot distinguish gene from homolog, as in the case of HBA1 and HBA2, where the normal combined CN of the two identical genes is 4, and a deletion in either gene leads to collective CN≤3. Note that, in principle, the CN analysis described herein could be applied even to high-depth whole-genome shotgun sequencing (i.e., without the use of probes for enrichment).

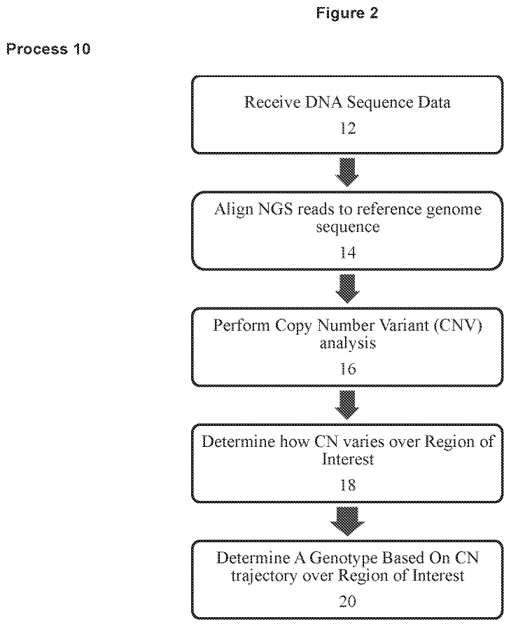

Broadly speaking, and in one example, to generate a call for a region, the following process is performed, which is illustrated as process 10 in FIG. 2. Initially, sequences of interest are obtained at 12. For example, reads can be collected from the bam file that overlap with the region of the call—or, critically, in the region(s) of its homolog(s)—in any way. These reads can then be clipped using their associated soft-clipping information. Auxiliary information from the aligner, e.g., base-to-base alignment information, can then be discarded, and the reads become simply a sequence of bases. (In some examples, filtering based on mapping quality can be optionally performed.)

Partition reads to gene or homolog(s) based on the presence of the base(s) that distinguish them. The distinguishing base(s) exploited in this partitioning process depend on the particular gene of interest. Further, the partitioning may only use a subset of the distinguishing bases in a given read, again based on the specific application. In an embodiment where the hybrid-capture probe sequence itself becomes part of the sequenced fragment, the hybrid-capture probe is designed such that the distinguishing base is at or near the terminus of one the ends of a paired-end read. For example in such a case, the hybrid-capture probe is, e.g., 39 bases long, but the sequencer reads 40 bases from the captured fragment. The probe is designed such that the 40th base is a distinguishing base, thereby allowing the entire read (i.e., both ends of the paired-end read) to be partitioned to gene or homolog(s) based on the 40th position's base. The precise numbers (i.e., 39 and 40) in the example above could change and yield similar results. In principle, the probe could be as short as 10 bp or as long as 1000 bp, though lengths in the range of 20 bp-100 bp are most common. In embodiments like the one above where the probe becomes part of the sequenced fragment, the sequencer must read beyond the length of the probe by at least 1 bp; however, in embodiments where the captured fragment alone contains enough distinguishing bases to partition the read appropriately to gene or homolog, then sequencing need not necessarily extend beyond the length of the probe.

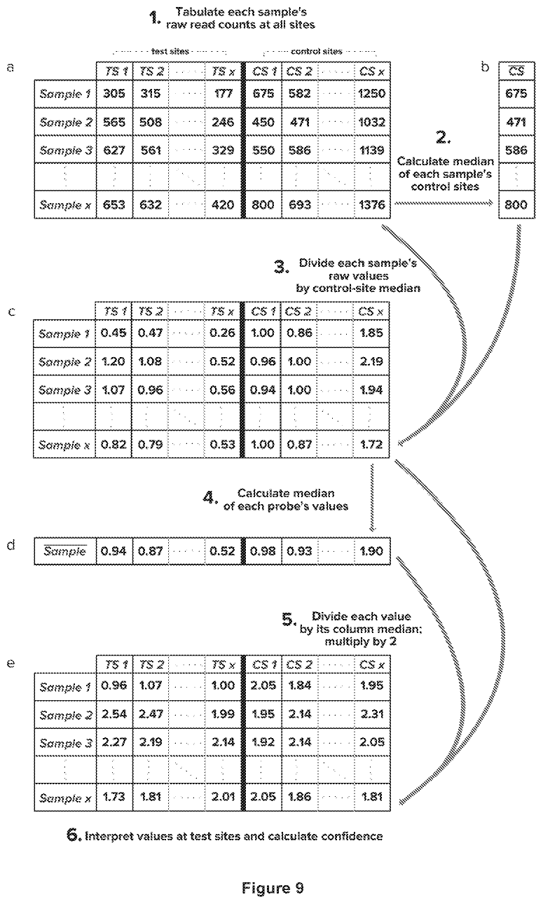

An exemplary treatment of experimental data is shown in FIG. 9. Shown is an excerpt from a table with data from a single experiment (using one Illumina flowcell). Each row is a sample. Typically, 48 or 96 samples are processed (i.e., tested) in a single experiment “Sample x”=“Sample 96”), though the analysis is valid for more or fewer samples. The analysis strongly leverages the fact that copy-number mutations are relatively rare, especially in genes associated with disease; thus, it is expected that the majority of samples will have the wild-type copy number (“CN”) at each site (i.e., CN=2).

As shown in FIG. 9, Table a, the sites can be partitioned into test sites (e.g., “TS1”, “TS2”, etc.) and control sites (e.g., “CS1”, “CS2”, etc.). The parsing of test sites (TS) versus control sites (CS) depends on the assay: for instance, in the Gaucher's disease assay, TS's are sites in GBA or GBAP, and CS's include any site in the genome for which we have data that is not in either GBA or GBAP. As another example, for the SMA test, there are only two TS sites (one for SMN1 and the other for SMN2). Typically, there are several hundred CS's for each experiment. If CN analysis is done in isolation, at least 10 CS's should be used, with 50 or more being preferable (basically, you need enough sites to get a robust measurement of the median, which we'll see in FIG. 9, Table b.)

The next step is depicted in FIG. 9, Table b, where the median for the CS raw read values are calculated. Note that each cell in the table could contain either integer-valued raw reads or floating point number of adjusted reads, where adjustments in read number could take into account factors like sequencing bias due to GC content. Note that this is only involving the CS's, since our initial assumption is that these values have CN=2; including TS's at this point could skew the median of a given row if the row's sample has a CN mutation and TS's outnumber OS's. Unlike using the mean to represent the average, the median is robust to outlier read values which are prevalent in sequencing data; however, you still should have at least 10 CS's to get a good representation of the median. This step is effectively performed by the following equation:

x_{i,j}=r_{i,j}/median(r_{i,CS1}:r_{i,CSX})

where r_{i,j} is the number of raw reads in sample i at site j. The median is evaluated over all sites j that are in the set of CS sites. x_{i,j} is the “sample-normalized depth value” for sample i at site j; x_{i,j} is calculated for all sites j in both CS and TS.

As provided for in FIG. 9, the value for each cell in Table a is divided by the corresponding value for the cell's row in Table b, and the quotient is written in Table c. Now the average value across a row is ˜1. However, further normalization is needed because there are site-specific biases in data acquisition that could corrupt our interpretation of the data. For instance, note how the values in the TSx column are systematically lower than the values in TS1 or TS2, Since it is implausible that this drop at TSx reflects a CN change in every sample (especially since the assumption is that CN variations are rare, thus it would be expected that such variations would not be in every sample), a further normalization is performed (in FIG. 9, Table d) to eliminate this systematic bias.

The normalization starts with calculating the median down each column. This is done for both TS and CS columns as shown in FIG. 9, Table d. Then, as shown in FIG. 9, Table e, the value for each cell in Table c is divided by the corresponding value for the cell's column in Table d; then the quotient is multiplied by two, and finally the product is written in Table e. We scale the quotient by 2 since division by the average gives a normalized value centered around 1, but we know that this normalized value corresponds to a biological normal CN of 2. This step is effectively performed by the following equation:

CN_{i,j}=2*x_{i,j}/median(x_{S1,j}:x_{SX,j})

where x_{i,j} is the “sample-normalized depth value” from above. The median is calculated over all samples for site j. CN_{i,j} is the decimal approximation of the copy number of site j in sample i. Since the copy number of a sequence in the genome is an integer value, each CN_{i,j} can be rounded to its nearest integer value, and confidence in the call can be calculated as described herein.

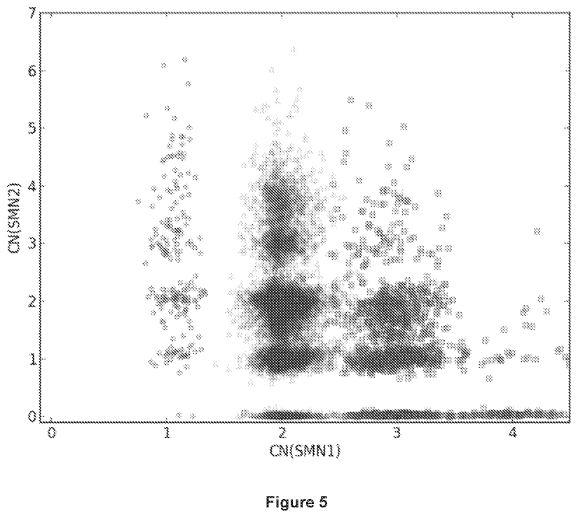

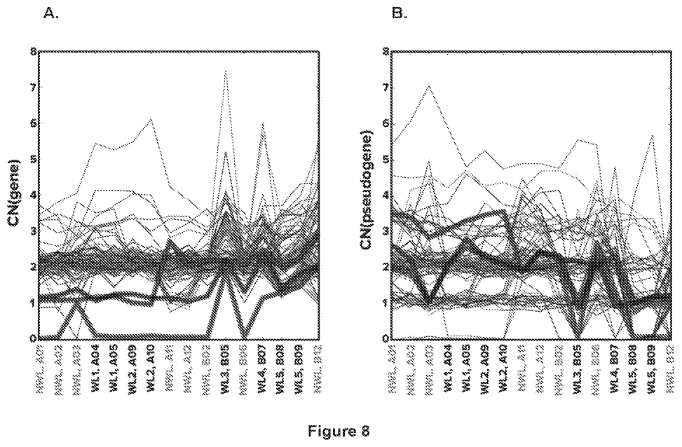

Note that the final normalization step indicated in the equation immediately above may be modified for TS's where CN is highly variable (i.e., where a small majority or even a minority of samples have CN=2), For instance, in the right plot of FIG. 8, the majority of samples have CN=0—not CN=2—for TS's “WL5,B08” and “WL5,B09”. We have encountered such TS's in analysis of SMA (FIG. 5) and CAH (FIG. 8). CN values at these challenging TS's can be determined by finding the best least-squares-deviation fit of a multimodal Gaussian distribution (with modes at empirically expected integer CN values, e.g., 0, 1, 2, and 3) to the empirically observed data, The CN value for each sample can then be determined by finding the minimum distance to an integer mode of the best-fit distribution.

The final step is interpretation of the data. For each disease—Congenital Adrenal Hypertrophy (CAH), Spinal Muscular Atrophy (SMA), Gaucher's, and alpha-thalassemia—we're looking for contiguous TS's in which the CN signal deviates from 2. Note that “Sample 1” in FIG. 9 has a CN value hovering around 1, unlike the other samples which have CN values centered at 2. These data suggest that Sample 1 has a CN mutation which has lowered its CN from two to one at the TS's. It's reassuring to see that Sample 1's CN values at CS's are ˜2, suggesting that the analysis was sound (i.e., it's not making the claim that the sample has a CN mutation everywhere in the genome, an implausibility).

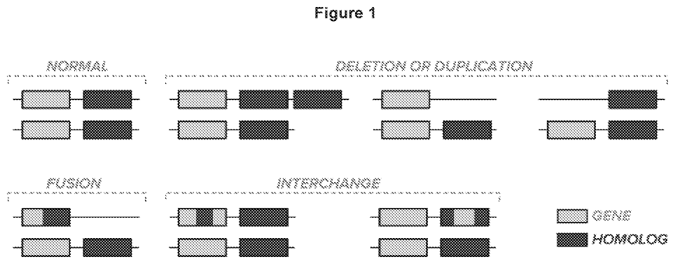

It is worth noting that the CN analysis described herein is a critical upstream step for finding other types of clinically relevant mutations in a gene with a homolog. For instance, in addition to CN variants (shown in FIG. 1), single-nucleotide polymorphisms (SNPs) may also disrupt a gene and render it dysfunctional. Standard software for recognizing SNPs uses CN as a parameter, where the expected fraction of reads bearing a SNP is 1/CN. Since most parts of the genome have CN=2, SNP-finding software by default identifies sites as SNPs when ½ of reads contain one base (e.g., C) and the other ½. have a different base (e.g., T), For regions with CN variation, however, the expected fraction of reads bearing a SNP could be 1 for CN=1, ⅓ for CN=3, and so on. Critically, in the absence of a CN analysis like the one described herein, a subject who has both a SNP and CN=3 may not have the SNP identified since its representation in the data (i.e., ⅓) would be less than the naively expected fraction (i.e., ½). Thus, the approach we describe herein is important not only for resolving genotype in terms of CN, but also in terms of finding other mutations like SNPs and short insertions/deletions (“indels”).

Since we typically have multiple TS's for a given test, we can assess confidence in our CN determination using a z-score. Here are the steps that may be used:
a. Calculate the interquartile range (“IQR”) for each TS column. The IQR is the difference between the 75^{th}- and 25^{th}-percentile values. Assuming normal-distribution statistics, convert the IQR to a standard deviation (“SD”) by dividing by ˜1.33. We use the IQR as an intermediate step to finding the SD, since IQR is insensitive to outliers whereas SD can shift wildly with outliers. This attention to outliers is especially important because the rare samples with CN mutations will effectively be outliers in each column.
b. With the SD in hand for each TS column, we next enumerate the hypotheses CN=1, CN=2, etc.), and for each hypothesis we determine how many SD's away from the hypothetical CN value our observed CN values are (this number of SD's from the assumed average value is the z-score). Next, we can convert z-scores to probabilities, which allow us to assess the likelihood of the hypothesis given the data. Treating each site as an independent observation, we calculate the probability across many TS's as the product of probabilities for each TS. Our confidence score is effectively a log-odds score, where we divide the probability of the highest-probability hypothesis by the probability of the second-highest probability hypothesis, and then take the log10 of this quotient.
   One of skill in the art will appreciate that other statistical approaches that are insensitive to outliers and yields an approximation of the standard deviation of the data may be used. Identification of spans of similar copy number (e.g., a series of adjacent sites with CN=1, consistent with a large deletion) can be identified in a supervised manner (e.g., by eye or by matching to known or hypothesized recombination sites) or unsupervised (e.g., using a Hidden Markov Model).

### Exemplary Architecture and Processing Environment:

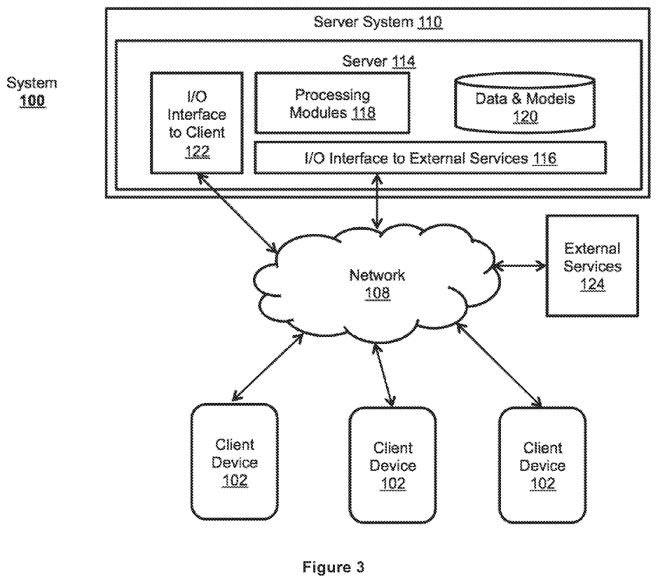

An exemplary environment and system in which certain aspects and examples of the systems and processes described herein may operate. As shown in FIG. 3, in some examples, the system can be implemented according to a client-server model. The system can include a client-side portion executed on a user device 102 and a server-side portion executed on a server system 110. User device 102 can include any electronic device, such as a desktop computer, laptop computer, tablet computer, PDA, mobile phone (e.g., smartphone), or the like.

User devices 102 can communicate with server system 110 through one or more networks 108, which can include the Internet, an intranet, or any other wired or wireless public or private network. The client-side portion of the exemplary system on user device 102 can provide client-side functionalities, such as user-facing input and output processing and communications with server system 110. Server system 110 can provide server-side functionalities for any number of clients residing on a respective user device 102. Further, server system 110 can include one or caller servers 114 that can include a client-facing I/O interface 122, one or more processing modules 118, data and model storage 120, and an I/O interface to external services 116. The client-facing I/O interface 122 can facilitate the client-facing input and output processing for caller servers 114. The one or more processing modules 118 can include various issue and candidate scoring models as described herein. In some examples, caller server 114 can communicate with external services 124, such as text databases, subscriptions services, government record services, and the like, through network(s) 108 for task completion or information acquisition. The I/O interface to external services 116 can facilitate such communications.

Server system 110 can be implemented on one or more standalone data processing devices or a distributed network of computers. In some examples, server system 110 can employ various virtual devices and/or services of third-party service providers (e.g., third-party cloud service providers) to provide the underlying computing resources and/or infrastructure resources of server system 110.

Although the functionality of the caller server 114 is shown in FIG. 3 as including both a client-side portion and a server-side portion, in some examples, certain functions described herein (e.g., with respect to user interface features and graphical elements) can be implemented as a standalone application installed on a user device. In addition, the division of functionalities between the client and server portions of the system can vary in different examples. For instance, in some examples, the client executed on user device 102 can be a thin client that provides only user-facing input and output processing functions, and delegates all other functionalities of the system to a backend server.

It should be noted that server system 110 and clients 102 may further include any one of various types of computer devices, having, e.g., a processing unit, a memory (which may include logic or software for carrying out some or all of the functions described herein), and a communication interface, as well as other conventional computer components (e.g., input device, such as a keyboard/touch screen, and output device, such as display). Further, one or both of server system 110 and clients 102 generally includes logic (e.g., http web server logic) or is programmed to format data, accessed from local or remote databases or other sources of data and content. To this end, server system 110 may utilize various web data interface techniques such as Common Gateway Interface (CGI) protocol and associated applications (or “scripts”), Java® “servlets,” i.e., Java® applications running on server system 110, or the like to present information and receive input from clients 102. Server system 110, although described herein in the singular, may actually comprise plural computers, devices, databases, associated backend devices, and the like, communicating (wired and/or wireless) and cooperating to perform some or all of the functions described herein. Server system 110 may further include or communicate with account servers (e.g., email servers), mobile servers, media servers, and the like.

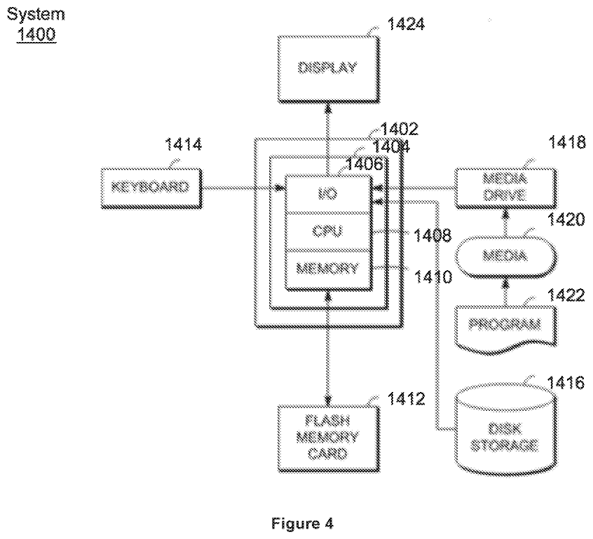

It should further be noted that although the exemplary methods and systems described herein describe use of a separate server and database systems for performing various functions, other embodiments could be implemented by storing the software or programming that operates to cause the described functions on a single device or any combination of multiple devices as a matter of design choice so long as the functionality described is performed. Similarly, the database system described can be implemented as a single database, a distributed database, a collection of distributed databases, a database with redundant online or offline backups or other redundancies, or the like, and can include a distributed database or storage network and associated processing intelligence. Although not depicted in the figures, server system 110 (and other servers and services described herein) generally include such art recognized components as are ordinarily found in server systems, including but not limited to processors, RAM, ROM, clocks, hardware drivers, associated storage, and the like (see, e.g., FIG. 4, discussed below). Further, the described functions and logic may be included in software, hardware, firmware, or combination thereof.

FIG. 4 depicts an exemplary computing system 600 configured to perform any one of the above-described processes, including the various calling and scoring models. In this context, computing system 600 may include, for example, a processor, memory, storage, and input/output devices (e.g., monitor, keyboard, disk drive, Internet connection, etc.). However, computing system 600 may include circuitry or other specialized hardware for carrying out some or all aspects of the processes. In some operational settings, computing system 600 may be configured as a system that includes one or more units, each of which is configured to carry out some aspects of the processes either in software, hardware, or some combination thereof.

FIG. 4 depicts computing system 600 with a number of components that may be used to perform the above-described processes. The main system 1402 includes a motherboard 1404 having an input/output (“I/O”) section 1406, one or more central processing units (“CPU”) 1408, and a memory section 1410, which may have a flash memory card 1412 related to it. The I/O section 1406 is connected to a display 1424, a keyboard 1414, a disk storage unit 1416, and a media drive unit 1418. The media drive unit 1418 can read/write a computer-readable medium 1420, which can contain programs 1422 and/or data.

At least some values based on the results of the above-described processes can be saved for subsequent use. Additionally, a non-transitory computer-readable medium can be used to store (e.g., tangibly embody) one or more computer programs for performing any one of the above-described processes by means of a computer. The computer program may be written, for example, in a general-purpose programming language (e.g., Pascal, C, C++, Python, Java) or some specialized application-specific language.

Various exemplary embodiments are described herein. Reference is made to these examples in a non-limiting sense. They are provided to illustrate more broadly applicable aspects of the disclosed technology. Various changes may be made and equivalents may be substituted without departing from the true spirit and scope of the various embodiments. In addition, many modifications may be made to adapt a particular situation, material, composition of matter, process, process act(s) or step(s) to the objective(s), spirit or scope of the various embodiments. Further, as will be appreciated by those with skill in the art, each of the individual variations described and illustrated herein has discrete components and features that may be readily separated from or combined with the features of any of the other several embodiments without departing from the scope or spirit of the various embodiments. All such modifications are intended to be within the scope of claims associated with this disclosure.

### EXAMPLES

The present invention is described in further detain in the following examples which are not in any way intended to limit the scope of the invention as claimed. The attached Figures are meant to be considered as integral parts of the specification and description of the invention. All references cited are herein specifically incorporated by reference for all that is described therein. The following examples are offered to illustrate, but not to limit the claimed invention.

### Example 1

### CALLING GENE/HOMOLOG COPY NUMBER

This example illustrates the method for determining gene/homolog copy number and is schematized in FIG. 9.

The method comprises the following steps.
1. Pooled all reads that BWA (an open-source computer software program that aligns NGS reads to a reference genome) assigned to gene or homolog(s).
2. Counted depth (i.e., the number of aligned reads) for gene and homolog, respectively (e.g., at the intronic position that distinguishes SMN1 from SMN2), based on the sequence of the read (optionally adjust read depth to take GC bias into account).
3. Tallied depth near 50 other control sites (“CS” in FIG. 9)
4. Normalized each sample's gene and homolog depths by the median of the sample's 50 control depths.
5. Further adjusted the data by normalizing by each site's median value, yielding a decimal-based copy-number value (e.g., 1.21).
6. Made copy-number calls (i.e., mapped decimal value from prior step to an integer value) based on statistical assessment of confidence.

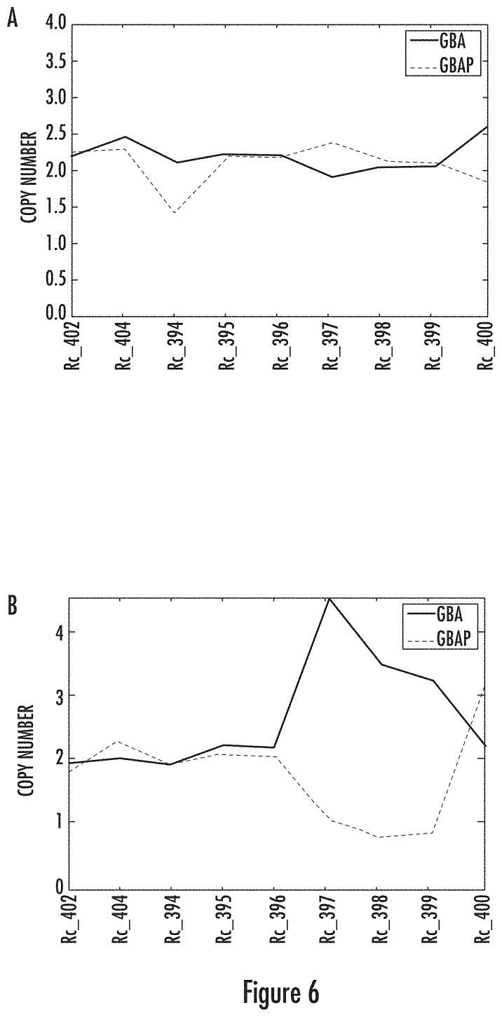

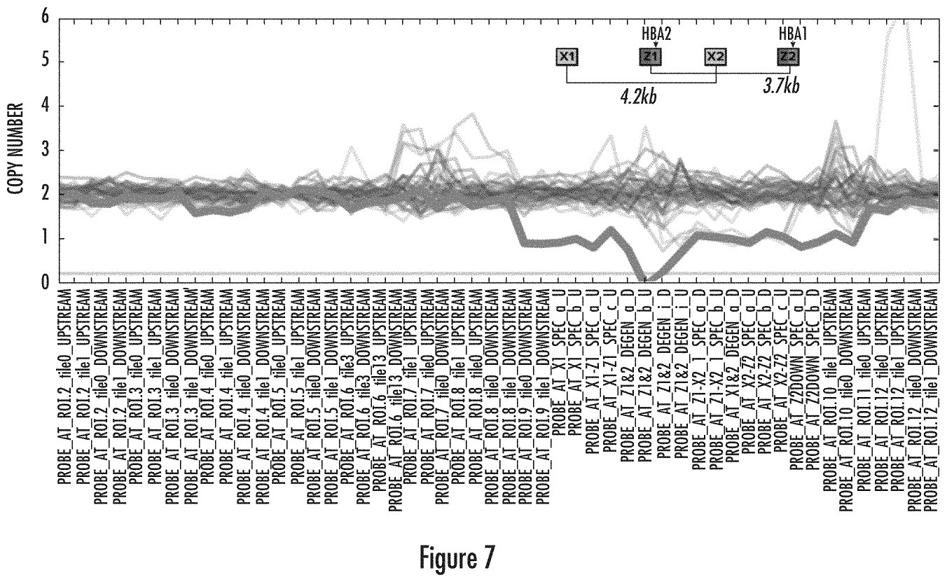

Results for various gene/homolog determinations are shown in FIGS. 5-8.

### Example 2

### COPY NUMBER ANALYSIS USING HYBRID-CAPTURE PROBES

This example illustrates the method for determining gene/homolog copy number for a specific gene using probes that anneal adjacent to a base that is different between the gene and the homolog(s) or pseudogene(s).

Hybrid-capture probes were designed to anneal adjacent to the few bases that differ between CYP21A2 and CYP21A1P (“diff bases”). Paired-end NGS of captured fragments allows designation of reads as being either gene- or pseudogene-derived based on the diff bases. CAH variants were identified using two strategies: SNP-based calling and copy-number analysis. SNP-based calling at a given position searched for deleterious and/or pseudogene-derived bases in a pileup composed of reads with gene-derived diff bases distal from the position of interest. By contrast, copy-number analysis used read depth of diff bases to calculate the relative abundance of each variant, and deleterious variants were identified as those with excess copy number of pseudogene-derived sequence (and, conversely, depleted copy number of gene-derived sequence). Long-range PCR and Sanger sequencing were used to confirm variants in a validation study.

The test correctly identified the genotypes of positive-control samples from affected patients, and we have since run the validated CAH test on nearly 150,000 clinical samples. The variant frequencies observed are consistent with prior studies that sequenced CYP21A2 in affected patients. There is great diversity in the copy number of gene and pseudogene: 38% of patients have at least one haplotype that does not simply have one copy of each. Evidence for recombination between gene and pseudogene is widespread, with at least 83% having a CYP21A2 haplotype containing pseudogene-derived bases. Finally, the test identifies compound variants consistent with specific rare haplotypes, e.g., (1) three copies of CYP21A2 where one has the Q319X mutation, and (2) CYP21A2 with a V282L mutation in cis with two copies of CYP21A1P, a haplotype enriched in Ashkenazi Jewish patients.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1-11. (canceled)

12. A method for determining a genotype of a genomic region in a genome sample, the method comprising:
a. Generating at least one probe that hybridizes to one or more homologous sites of interest in a genome sample;
i. wherein said one or more sites of interest comprise at least a first test site and a second test site;
ii. wherein said at least first test site comprises at least a portion of the nucleic acid sequence of a target genomic region and said at least second test site comprises at least a portion of the nucleic acid sequence of a homolog or pseudogene of said target genomic region;
iii. wherein said at least one probe is n bases long and captures a distinguishing fragment comprising at least one distinguishing base that distinguishes said one or more homologous sites of interest;
b. Annealing said at least one probe to said one or more homologous sites of interest in the genome sample;
c. Enriching the genome samples of b by obtaining sequence reads enriched for said distinguishing fragment;
d. Obtaining sequence reads for one or more control sites in the genome sample;
e. Aligning the sequence reads from steps c and d to a human reference genome;
f. Partitioning in silico the sequence reads from steps c and d to first test site, second test site, or control site based on alignment to the human reference genome and the presence or absence of said at least one distinguishing base;
g. Counting the number of reads partitioned in step f;
h. Performing copy number analysis that converts the raw number of reads counted into interpretable copy-number calls; and
i. Determining the genotype of the genomic region based on the copy number analysis.

13. The method of claim 12 wherein the sequencer reads n+x bases in step c, x is greater than or equal to 1, and said at least one distinguishing base is proximate to an end of said sequence reads.

14. The method of claim 13, wherein the probe is designed such that the n+x^{th} base is a distinguishing base, thereby allowing the entire read to be partitioned to the first test site or the second test site based on the n+x^{th} base's position.

15. The method of claim 12, wherein the sequence reads are obtained using Next Generation Sequencing (NGS).

16. The method of claim 15, wherein the NGS comprises targeted DNA sequencing with hybrid-capture technology.

17. The method of claim 12, wherein the probes are specifically designed to yield reads unique to either the first test site, the second test site, or a control site.

18. The method of claim 12, wherein the probes are specifically designed such that captured and sequenced fragments contain at least one sequence that distinguish the first test site from the second test site.

19. The method of claim 12, wherein said sequence reads in step d comprise sequence reads from multiple control sites in a genome sample, wherein the sequence reads are obtained from two or more control sites, ≥10 control sites, ≥50 control sites, or several hundred control sites.

20. The method of claim 12, wherein step g comprises counting the number of reads both at the sites of interest and the multiple control sites.

21. The method of claim 12, further comprising step j:
i. Identifying mutations, the mutations comprising copy-number variants, inversions that alter orientation, gene fusions, and/or short sequence variants.

22. The method of claim 21, wherein the short sequence variants comprise SNPs and/or indels.

23. The method of claim 12, wherein the analysis in step h comprises determining a copy number at the sites of interest or the one or more control sites by normalizing the raw number of reads obtained at the sites of interest or the one or more control sites.

24. The method of claim 12, wherein the sequencer reads beyond the length of the at least one probe by at least 1 bp.

25. The method of claim 12, wherein the first test site and the second test site are associated with a gene and its homolog, and wherein the gene is HBA1 and the homolog is HBA2, or the gene is HBA2 and the homolog is HBA1.

26. The method of claim 12, wherein the first test site and the second test site are associated with a gene and its homolog.

27. The method of claim 12, wherein the first test site and the second test site are associated with a gene and its pseudogene.

28. The method of claim 12, wherein the analysis is step h comprises:
i. Calculating a median read number for the raw number of reads at the one or more control sites for each sample;
ii. Dividing the raw number of reads counted both at the sites of interest and the one or more control sites of each sample by the median read number calculated in step (i), thereby calculating a first normalized read count both at the sites of interest and the one or more control sites for each sample;
iii. Calculating a median first normalized read count for each site of interest and control site for all samples; and
iv. Dividing the first normalized read count both at the sites of interest and the one or more control sites of each sample by the corresponding median first normalized read count calculated in step (iii), thereby calculating a second normalized read count both at the sites of interest and the one or more control sites for each sample.

29. The method of claim 28, comprising multiplying the second read count calculated in step iv by 2 to calculate a copy number at a site of interest or control site.

30. The method of claim 28, comprising finding the best least-squares-deviation fit of a multimodal Gaussian distribution to variable read counts calculated at a site of interest in step iv and determining the copy number at the site of interest by finding the minimum distance to an integer mode of the best-fit distribution.

31. The method of claim 30, wherein the copy number calculated is rounded to its nearest integer value.

32. The method of claim 30, further comprising calculating a confidence score for the copy number calculated.

33. The method of claim 32, wherein the confidence score is calculated using a z-score.

34. The method of claim 28, comprising providing multiple genome samples and performing steps a-g of claim 12 on each sample before step i of claim 66.

35. The method of claim 34, wherein 48 or more than 48 genome samples are provided.

36. The method of claim 34, wherein 96 or fewer than 96 genome samples are provided.

37. The method of claim 12, wherein the first test site and the second test site are associated with a gene and its homolog, and wherein the gene is SMN1 and the homolog is SMN2, the gene is CYP21A2 and the homolog is CYP21A1P, the gene is HBA1 and the homolog is HBA2, the gene is HBA2 and the homolog is HBA1, the gene is GBA and the homolog is GBAP, the gene is CHEK2 and the homolog is at least one of its pseudogenes, or the gene is PMS2 and the homolog is selected from PMS2CL and its other pseudogenes.

38. The method of claim 12, wherein the partitioning in step f only uses a subset of the distinguishing bases in a given read.

39. The method of claim 12, wherein the at least one probes are between 10 bp to 1000 bp in length.

40. The method of claim 12, wherein the at least one probes are between 20 bp to 100 bp in length.

41. The method of claim 12, wherein the at least one probes are designed to anneal adjacent to the few bases that differ between the first test site and the second test site.

42. The method of claim 12, wherein a captured fragment alone contains enough distinguishing bases to partition the read appropriately to either the first test site or the second test site, and the sequencing does not extend beyond the length of a hybrid-capture probe.

43. The method of claim 15, wherein the NGS comprises targeted DNA sequencing with hybrid-capture technology using hybrid-capture probes, a captured fragment alone contains enough distinguishing bases to partition the read appropriately to either the first test site or the second test site, and the sequencing does not extend beyond the length of a hybrid-capture probe.

44. The method of claim 15, wherein the NGS comprises amplicon sequencing using primers that are specifically designed to yield reads unique to either the first test site or the second test site.

45. A method for determining a genotype of a genomic region in a genome sample, the method comprising:
a. Generating at least one hybrid capture probe for use in targeted DNA sequencing with hybrid capture technology that hybridizes to one or more sites of interest in the genome sample comprising at least a first test site and a second test site;
i. wherein said first test site comprises at least a portion of the DNA sequence of a gene of interest and said second test site comprises at least a portion of the DNA sequence of a homolog or pseudogene of said target gene; and
ii. wherein said hybrid capture probe is n bases long;
b. Annealing said at least one hybrid-capture probe to said sites of interest;
c. Capturing at least one distinguishing fragment from said sites of interest with said at least one hybrid-capture probe wherein said distinguishing fragment comprises at least one base that distinguishes said first test site and said second test site;
d. Amplifying said at least one distinguishing fragment and generating an enriched nucleic acid sample enriched for fragments comprising at least one base that distinguishes said first test site and said second test site;
e. Obtaining sequence reads experimentally from said enriched nucleic acid sample wherein said sequence reads are enriched for said at least one distinguishing fragment comprising at least one base that distinguishes said first test site and said second test site;
f. Aligning the sequence reads from step e to a human reference genome;
g. Partitioning in silico the sequence reads from step e to first test site or second test site based on alignment to a human reference genome and said at least one distinguishing base;
h. Counting the number of reads partitioned in step g;
i. Performing copy number analysis that converts the raw number of reads counted into interpretable copy-number calls; and
j. Determining the genotype of the genomic region based on the copy number analysis.

46. The method of claim 45 wherein the sequencer reads n+x bases from the captured fragment, x≥1, and said at least one distinguishing base is proximate to an end of said sequence reads.
